(19) United States
(12) Patent Application Publication
(10) Pub. No.: US 2010/0087610 A1
(43) Pub. Date: Apr. 8, 2010

(54) Method Of Preparing And Using A Molecular Sieve

(76) Inventors: Stephen N. Vaughn, Kingwood, TX (US); Machteld M. Mertens, Boortmeerbeek (BE); Thomas H. Colle, Houston, TX (US)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 12/533,745

(22) Filed: Jul. 31, 2009

Related U.S. Application Data
(60) Provisional application No. 61/103,050, filed on Oct. 6, 2008.

(51) Int. Cl. C08F 4/52; C01B 37/08; C07C 1/00

(52) U.S. Cl. 526/226; 423/305; 585/638

(57) ABSTRACT

A crystalline microporous silicoaluminophosphate (SAPO) molecular sieve is prepared by: (a) providing a reaction mixture having a molar composition within the following ranges: P:Al from 0.75 to 1.25, Si:Al₂ from 0.01 to 0.32, H₂O:Al₂ from 25 to 50, and R:Al₂ from 1 to 3, where R designates a structure directing agent (template), and wherein the molar ratios for the aluminum, phosphorus, and silicon sources are calculated based on the oxide forms; (b) heating the mixture to a crystallization temperature of between 150° C. to 250° C. to form the crystalline molecular sieve within the reaction mixture; and (c) following crystallization in step (b), aging the crystalline molecular sieve in an alkaline liquid at a temperature below the crystallization temperature from 0.5 hour for up to 120 hours. Aging can affect both catalyst life and catalyst activity.

### CROSS-REFERENCE TO RELATED APPLICATIONS

The application claims priority from U.S. Provisional Application Ser. No. 61/103,050, filed Oct. 6, 2008, the contents of which are incorporated by reference in its entirety.

### FIELD OF THE INVENTION

This invention relates to a method of preparing a molecular sieve, particularly but not exclusively to a method of preparing a crystalline microporous metalloaluminophosphate (ELAPO) molecular sieve, more in particular to the synthesis of a silicoaluminophosphate molecular sieve containing CHA framework. The molecular sieve of the invention is suitable for the conversion of oxygenates, particularly methanol, to olefins, particularly ethylene and/or propylene.

### BACKGROUND OF THE INVENTION

Olefins are traditionally produced from petroleum feedstock by catalytic or steam cracking processes. These cracking processes, especially steam cracking, produce light olefins such as ethylene and/or propylene from a variety of hydrocarbon feedstock. It has been known for some time that oxygenates, especially alcohols, e.g. methanol, are convertible into light olefins. The preferred methanol conversion process is generally referred to as a methanol-to-olefins process, where methanol is converted to primarily ethylene and propylene in the presence of a molecular sieve.

Some of the most useful molecular sieves for converting methanol to olefins are the metalloaluminophosphates such as the silicoaluminophosphates (SAPO's). There are a wide variety of SAPO molecular sieves known in the art. Of these the more important examples include SAPO-5, SAPO-11, SAPO-18, SAPO-34, SAPO-35, SAPO-41, and SAPO-56. For the methanol-to-olefins process SAPO molecular sieves having the CHA framework and especially SAPO-34 are particularly important catalysts. The CHA framework type has a double six-ring structure in an ABC stacking arrangement. The pore openings of the structure are defined by eight member rings that have a diameter of about 4.0 Å, and cylindrical cages within the structure of approximately 10×6.7 Å (“Atlas of Zeolite Framework Types”, 2001, 5th Edition, p. 96). Other SAPO molecular sieves of CHA framework type include SAPO-44, SAPO-47 and ZYT-6.

The synthesis of SAPO molecular sieves is a complicated process. There are a number of variables that need to be controlled in order to optimize the synthesis in terms of purity, yield and quality of the SAPO molecular sieve produced to arrive at a low Si/Al molecular sieve having a low silica to alumina molar ratio and a relatively small crystal size of typically less than 4 μm, preferably less than 1 μm.

A particularly important variable is the choice of synthesis template, which usually determines which SAPO framework type is obtained from the synthesis.

U.S. Pat. No. 6,793,901 discloses a method for preparing a microporous silicoaluminophosphate molecular sieve having the CHA framework type, which process comprises (a) forming a reaction mixture comprising a source of aluminum, a source of silicon, a source of phosphorus, optionally at least one source of fluoride ions and at least one template containing one or more N,N-dimethylamino moieties, (b) inducing crystallization of the silicoaluminophosphate molecular sieve from the reaction mixture, and (c) recovering silicoaluminophosphate molecular sieve from the reaction mixture. Suitable templates are said to include one or more of N,N-dimethylethanolamine, N,N-dimethylbutanolamine, N,N-dimethylheptanolamine, N,N-dimethylhexanolamine, N,N-dimethylethylenediamine, N,N-dimethylpropylenediamine, N,N-dimethylbutylenediamine, N,N-dimethylheptylenediamine, N,N-dimethylhexylenediamine, or dimethylethylamine, dimethylpropylamine, dimethylheptylamine and dimethylhexylamine. When conducted in the presence of fluoride ions, the synthesis is effective in producing low silica silicoaluminophosphate molecular sieves having a Si/Al atomic ratio of from 0.01 to 0.1. In the Examples of this patent, crystallization is conducted by heating the reaction mixture to 170° C. to 180° C. for 1 to 5 days.

U.S. Pat. No. 6,835,363 discloses a process for preparing microporous crystalline silicoaluminophosphate molecular sieves of CHA framework type, the process comprising: (a) providing a reaction mixture comprising a source of alumina, a source of phosphate, a source of silica, hydrogen fluoride and an organic template comprising one or more compounds of formula (I):

(CH₃)₂N—R—N(CH₃)₂ (I)

where R is an alkyl radical of from 1 to 12 carbon atoms; (b) inducing crystallization of silicoaluminophosphate from the reaction mixture; and (c) recovering silicoaluminophosphate molecular sieve. Suitable templates are said to include one or more of the group consisting of: N,N,N′,N′-tetramethyl-1,3-propane-diamine, N,N,N′,N′-tetramethyl-1,4-butanediamine, N,N,N′,N′-tetramethyl-1,3-butanediamine, N,N,N′,N′-tetramethyl-1,5-pentanediamine, N,N,N′,N′-tetramethyl-1,6-hexanediamine, N,N,N′,N′-tetramethyl-1,7-heptanediamine, N,N,N′,N′-tetramethyl-1,8-octanediamine, N,N,N′,N′-tetramethyl-1,9-nonanediamine N,N,N′,N′-tetramethyl-1,10-decanediamine, N,N,N′,N′-tetramethyl-1,11-undecanediamine and N,N,N′,N′-tetramethyl-1,12-dodecanediamine. In the Examples, crystallization is conducted by heating the reaction mixture to 120 to 200° C. for 4 to 48 hours.

U.S. Pat. No. 7,247,287 discloses the synthesis of silicoaluminophosphate molecular sieves having the CHA framework type employing a directing agent having the formula (II):

R¹R²N—R³ (II)

wherein R¹ and R² are independently selected from the group consisting of alkyl groups having from 1 to 3 carbon atoms and hydroxyalkyl groups having from 1 to 3 carbon atoms and R³ is selected from the group consisting of 4- to 8-membered cycloalkyl groups, optionally substituted by 1 to 3 alkyl groups having from 1 to 3 carbon atoms; and 4- to 8-membered heterocyclic groups having from 1 to 3 heteroatoms, said heterocyclic groups being optionally substituted by 1 to 3 alkyl groups having from 1 to 3 carbon atoms and the heteroatoms in said heterocyclic groups being selected from the group consisting of O, N, and S. Preferably, the directing agent is selected from N,N-dimethyl-cyclohexylamine, N,N-dimethyl-methyl-cyclohexylamine, N,N-dimethyl-cyclopentylamine, N,N-dimethyl-methyl-cyclopentylamine, N,N-dimethyl-cycloheptylamine, N,N-dimethyl-methylcycloheptylamine, and most preferably is N,N-dimethyl-cyclohexylamine.

WO 03/035549 discloses a method for the manufacture of SAPO sieves which includes the step of maintaining the slurry of the as crystallized molecular sieve under substantially static conditions when stored after substantially complete crystallization and prior to recovery of the product. In the context of WO 03/035549, “substantially static” means that the slurry is maintained in an unstirred, not agitated condition for at least 80% of the time, whilst short bursts of low impact agitation are disclosed to prevent compaction of the crystalline molecular sieve at the bottom of the storage vessel. According to the disclosure in WO 03/035549, storage under non-static conditions reduces the yield of synthesized crystalline molecular sieve and is therefore undesirable.

In the commercial operation of the sieve catalyst in an MTO process, an important consideration is that the sieve catalyst can be manufactured at competitive yields and/or that the manufacturing cost is reduced. Other important variables in the commercial operation are the selectivity for propylene and ethylene over other olefins (prime olefin selectivity or POS) and the catalyst lifetime.

The present invention seeks to provide an improved molecular sieve with respect to one or more of the above parameters, and/or to provide improvements generally.

### SUMMARY OF THE INVENTION

According to the invention there is provided a method as defined in any of the accompanying claims.

In an embodiment of the invention there is provided a method of preparing a crystalline microporous silicoaluminophosphate (SAPO) molecular sieve comprising:
(a) providing a reaction mixture having a molar composition within the following ranges:
   P:Al from 0.75 to 1.25,
   Si:Al₂ from 0.01 to 0.32,
   H₂O:Al₂ from 25 to 50, and
   R:Al₂ from 1 to 3,
      where R designates the structure directing agent (template), and wherein the molar ratios for the aluminum, phosphorus, and silicon sources are calculated based on the oxide forms;
(b) heating the mixture to a crystallization temperature of between 150° C. to 250° C. to form the crystalline molecular sieve within the reaction mixture; and
(c) following crystallization step b) during stirring, aging the crystalline molecular sieve in an alkaline liquid at a temperature below the crystallization temperature from 0.5 hour for up to 120 hours.

Aging during stirring in an aging medium in the form of an alkaline liquid can improve both the catalyst life [expressed as CMCPS, or grams of reactant (in this case, an oxygenate such as methanol) converted per gram of molecular sieve, before the reactant drops below 10% conversion] and the POS when the sieve/catalyst is subsequently used in an oxygenates to olefins conversion process. Where “POS” is defined as (g of ethylene+propylene) produced per g of MeOH (excluding H2O) times 100%. The crystalline molecular sieve can be aged after it has been crystallized at a crystallization temperature. Typically, the temperature at which the crystalline molecular sieve is aged is below the temperature at which the reaction mixture has been heated to form the crystalline molecular sieve. Preferably, the crystalline molecular sieve is aged below a crystallization temperature of the reaction mixture. Stirring in the context of this application means agitating the crystalline molecular sieve beyond a level which is necessary to prevent compaction of the sieve crystals during storage. This level is higher than the level which is disclosed in WO 03/035549.

In a preferred embodiment, the alkaline liquid comprises the reaction mixture (“aging in the mother liquor”). This is particularly advantageous as no additional aging medium is required. Following aging, the sieve may be dried. Prior to drying, the sieve may be washed.

In a particular embodiment, the sieve is aged in a washing fluid. Alternatively, the sieve may be spray dried in the presence of a binder to make formulated catalyst particles and the sieve is aged in a slurrying fluid. The aging medium may thus comprise a slurrying fluid and/or a washing fluid. In effect, the synthesized sieve can be re-dispersed in an aging medium and, following aging, the sieve can be dried.

In a further embodiment, the synthesized sieve may be aged post synthesis by re-dispersing the sieve in an alkaline liquid and aging the sieve in the liquid.

In a preferred embodiment, the molecular sieve is washed and dried following heating step b) and prior to aging step c). Aging of the synthesis mixture following heating of the mixture and before washing and drying (“aging in the mother liquor”) can advantageously improve the catalyst life and/or the POS when the sieve/catalyst is subsequently used in an oxygenates to olefins conversion process. In other words, stirring while aging can reduce the aging time necessary to achieve improved catalyst life and/or POS. The mixture may be aged from 0.5 hour to 120 hours, for example between 2 and 5 days. The mixture is typically aged at a temperature below the crystallization temperature, preferably at a temperature between 10° C. and 100° C., such as between 20° C. and 70° C.

In a preferred embodiment, the mixture is aged whilst stirring continuously. This can have the advantage that an improved catalyst life and/or POS can be achieved without prolonged aging. If the synthesis mixture is aged whilst stirring continuously, the aging time may be reduced to 25 days or less, typically 10 days or less, and more typically 5 days or less. Another advantage can occur when the crystals present in the mother liquor are prevented from settling during the aging process. If the crystals form a dense cake during the aging process, the recovery may be more difficult.

The mixture may be aged at room temperature. As used herein, the term “room temperature” is meant to indicate a temperature of roughly 20° C. to 25° C. Alternatively, the mixture may be aged at an elevated temperature which is higher than room temperature, but lower than the crystallization temperature. In another alternative embodiment, the mixture is aged at a temperature below room temperature.

In a preferred embodiment, prior to aging, the crystallized molecular sieve has a crystal size greater than 1 μm, for example greater than 2 μm, preferably 2.5 μm or greater than 3.0 μm. It has been found that aging can have a more pronounced effect on the POS and/or the catalyst life for molecular sieves which have a crystal size of greater than 3.0 μm. The crystallized molecular sieve may exhibit a crystal size between 0.5 μm and 5 μm, such as between 1 μm and 4 μm.

The sources of aluminum, phosphorus, and silicon suitable for use in the present synthesis method are typically those known in the art or as described in the literature for the production of aluminophosphates and silicoaluminophosphates. For example, the aluminum source may be an aluminum oxide (alumina), optionally hydrated, an aluminum salt, especially a phosphate, an aluminate, or a mixture thereof. Other sources may include alumina sols or organic alumina sources, e.g., aluminum alkoxides such as aluminum isopropoxide. A preferred source is a hydrated alumina, most preferably pseudoboehmite, which contains 75% Al₂O₃ and 25% H₂O by weight. Typically, the source of phosphorus is a phosphoric acid, especially orthophosphoric acid, although other phosphorus sources, for example, organophosphates (e.g., trialkylphosphates such as triethylphosphate) and aluminophosphates may be used. When organophosphates and/or aluminophosphates are used, typically they are present collectively in a minor amount (i.e., less than 50% by weight of the phosphorus source) in combination with a majority (i.e., at least 50% by weight of the phosphorus source) of an inorganic phosphorus source (such as phosphoric acid). Suitable sources of silicon include silica, for example colloidal silica, such as one sold by EI du Pont de Nemours under the tradename Ludox, and fumed silica, as well as organic silicon source, e.g., a tetraalkyl orthosilicate, preferably such as tetraethylorthosilicate (TEOS), tetramethylorthosilicate (TMOS), or the like, or a combination thereof.

Although, in most embodiments, the sources of silicon, phosphorus, and aluminum are the only components that form the framework of a calcined silicoaluminophosphate molecular sieve according to the invention, it is possible for some small portion (e.g., typically no more than 10 wt %, such as no more than 5 wt %) of the silicon source can be substituted with a source of one or more of magnesium, zinc, iron, cobalt, nickel, manganese, and chromium.

In an embodiment, the reaction mixture has a molar composition within the following ranges:
P:Al from 0.75 to 1.25,
Si:Al₂ from 0.01 to 0.32,
H₂O:Al₂ from 25 to 50, and
R:Al₂ from 1 to 3,
   where R designates the structure directing agent (template), and wherein the molar ratios for the aluminum, phosphorus, and silicon sources are calculated based on the oxide forms, regardless of the form of the source added to the reaction mixture (e.g., whether the phosphorus source is added to the reaction mixture as phosphoric acid, H₃PO₄, or as triethylphosphate, the molar ratio is normalized to P₂O₅ molar equivalents).

Although the reaction mixture may also contain a source of fluoride ions, it is found that the present synthesis will proceed in the absence of fluoride ions, and hence it is generally preferred to employ a reaction mixture which is substantially free of fluoride ions.

In another embodiment of the invention, there is provided a method for converting hydrocarbons into olefins comprising the steps of preparing a molecular sieve as hereinbefore described, preparing a catalyst composition comprising said sieve, contacting said catalyst composition with a hydrocarbon feed under condition sufficient to convert a hydrocarbon feed into an olefin product, the conditions preferably comprising a temperature ranging from 200° C. to 1000° C. and a pressure ranging from 0.1 kPaa to 5 MPaa, as disclosed in detail in U.S. Pat. No. 5,952,553, which is herein fully incorporated by reference.

The hydrocarbon feed may comprise an oxygenate feed comprising methanol, dimethyl ether, or a combination thereof. The olefins may comprise ethylene, propylene, or a combination thereof.

In another embodiment, there is provided a method of forming an olefin-containing polymer product comprising preparing a molecular sieve as herein before described, formulating a catalyst comprising said molecular sieve, contacting said catalyst with a hydrocarbon feed under conditions to convert the hydrocarbon feed into one or more olefins; and polymerizing at least one of the one or more olefins, optionally with one or more comonomers, and optionally in the presence of a polymerization catalyst to form an olefin-containing polymer. The olefin containing polymer may comprise an ethylene containing polymer, a propylene containing polymer, and/or a copolymer mixture or blend thereof.

### BRIEF DESCRIPTION OF THE DRAWINGS

The invention will now be described in further detail, by way example only and with reference to the accompanying drawings in which

FIG. 1 presents a diagram showing the relationship between catalyst life and static aging according to another embodiment of the invention;

FIG. 2 presents a diagram showing the relationship between catalyst yield and static aging according to an embodiment of the invention;

FIG. 3 presents a diagram showing the relationship between POS and aging according to an embodiment of the invention; and

FIG. 4 presents a diagram showing the relationship between catalyst life and stirred aging according to an embodiment of the invention.

### DETAILED DESCRIPTION OF SPECIFIC EMBODIMENTS

Described herein is a method of synthesizing a crystalline aluminophosphate or silicoaluminophosphate containing a CHA framework type molecular sieve and to the use of the resultant molecular sieve as a catalyst in organic conversion reactions, especially the conversion of oxygenates to light olefins. In particular, it has been found that if a reaction mixture is aged following crystallization of the molecular sieve but before the molecular sieve is extracted from the reaction mixture and washed and dried, and aging takes place at a temperature which is below the crystallization temperature, a CHA framework-type containing molecular sieve is produced with improved prime olefin selectivity (POS) and improved catalyst life.

In the present method, a reaction mixture is prepared comprising a source of aluminum, a source of phosphorous, at least one organic directing agent, and, optionally, a source of silicon. Any organic directing agent capable of directing the synthesis of CHA framework type molecular sieves can be employed, but generally the directing agent is a compound having the formula (III):

R¹R²N—R³ (III)

wherein R¹ and R² independently comprise alkyl groups having from 1 to 3 carbon atoms or hydroxyalkyl groups having from 1 to 3 carbon atoms, and wherein R³ comprises a 4- to 8-membered cycloalkyl group, optionally substituted by 1 to 3 alkyl groups each having from 1 to 3 carbon atoms, or a 4- to 8-membered heterocyclic groups having from 1 to 3 heteroatoms each selected from the group consisting of O, N, and S, said heterocyclic groups being optionally substituted by 1 to 3 alkyl groups having from 1 to 3 carbon atoms.

More particularly, the organic directing agent is a compound having the formula (IV):

(CH₃)₂N—R³ (IV)

wherein R³ is a 4- to 8-membered cycloalkyl group, especially a cyclohexyl group, optionally substituted by 1 to 3 methyl groups. Particular examples of suitable organic directing agents include, but are not limited to, at least one of N,N-dimethyl-cyclohexylamine, N,N-dimethyl-methylcyclohexylamine, N,N-dimethyl-cyclopentylamine, N,N-dimethyl-methylcyclopentylamine, N,N-dimethyl-cycloheptylamine, and N,N-dimethyl-methylcycloheptylamine, especially N,N-dimethyl-cyclohexylamine.

The sources of aluminum, phosphorus, and silicon suitable for use in the present synthesis method are typically those known in the art or as described in the literature for the production of aluminophosphates and silicoaluminophosphates. For example, the aluminum source may be an aluminum oxide (alumina), optionally hydrated, an aluminum salt, especially a phosphate, an aluminate, or a mixture thereof. Other sources may include alumina sols or organic alumina sources, e.g., aluminum alkoxides such as aluminum isopropoxide. A preferred source is a hydrated alumina, most preferably pseudoboehmite, which contains 75% Al₂O₃ and 25% H₂O by weight. Typically, the source of phosphorus is a phosphoric acid, especially orthophosphoric acid, although other phosphorus sources, for example, organophosphates (e.g., trialkylphosphates such as triethylphosphate) and aluminophosphates may be used. When organophosphates and/or aluminophosphates are used, typically they are present collectively in a minor amount (i.e., less than 50% by weight of the phosphorus source) in combination with a majority (i.e., at least 50% by weight of the phosphorus source) of an inorganic phosphorus source (such as phosphoric acid). Suitable sources of silicon include silica, for example colloidal silica and fumed silica, as well as organic silicon source, e.g., a tetraalkyl orthosilicate.

Although, in most embodiments, the sources of silicon, phosphorus, and aluminum are the only components that form the framework of a calcined silicoaluminophosphate molecular sieve according to the invention, it is possible for some small portion (e.g., typically no more than 10 wt %, such as no more than 5 wt %) of the silicon source can be substituted with a source of one or more of magnesium, zinc, iron, cobalt, nickel, manganese, and chromium.

Although the reaction mixture may also contain a source of fluoride ions, it is found that the present synthesis will proceed in the absence of fluoride ions and hence it is generally preferred to employ a reaction mixture which is substantially free of fluoride ions.

Typically, the reaction mixture also contains seeds to facilitate the crystallization process. The amount of seeds employed can vary widely, but generally the reaction mixture comprises from 0.01 ppm by weight to 10,000 ppm by weight, such as from 100 ppm by weight to 5,000 by weight, of said seeds. Generally, the seeds can be homostructural with the desired product, that is are of a CHA framework type material, although heterostructural seeds of, for example, an AEI, LEV, ERI, AFX, or OFF framework-type molecular sieve, or a combination or intergrowth thereof, may be used. The seeds may be added to the reaction mixture as a suspension in a liquid medium, such as water; in some cases, particularly where the seeds are of relatively small size, the suspension can be colloidal. The production of colloidal seed suspensions and their use in the synthesis of molecular sieves are disclosed in, for example, International Publication Nos. WO 00/06493 and WO 00/06494, both published on Feb. 10, 2000 and both of which are incorporated herein by reference.

Crystallization of the reaction mixture can be carried out at either static or stirred conditions in a suitable reactor vessel, such as for example, polypropylene jars or Teflon-lined or stainless steel autoclaves. The crystallization regime can involve heating the reaction mixture relatively quickly, at a rate of more than 10° C./hour, conveniently at least 15° C./hour or at least 20° C./hour, for example from 15° C./hour to 150° C./hour or from 20° C./hour to 100° C./hour, to the desired crystallization temperature, typically between 50° C. and 250° C., for example from 150° C. to 225° C. or from 150° C. to 200° C., such as from 160° C. to 195° C. In some embodiments, however, the desired crystallization temperature is additionally at least 165° C., for example at least 170° C., and can optionally also be not more than 190° C., for example not more than 185° C. or not more than 180° C. In any of these embodiments, when the desired crystallization temperature is reached, the crystallization can be terminated immediately or from 5 minutes to 350 hours, and the reaction mixture can be allowed to cool; additionally or alternately, the crystallization can run for at least 12 hours, preferably at least 16 hours, for example at least 24 hours, at least 36 hours, at least 48 hours, at least 60 hours, at least 72 hours, at least 84 hours, at least 96 hours, at least 120 hours, or at least 144 hours before cooling. Additionally in this embodiment, on cooling, the crystalline product can be recovered by standard means, such as by centrifugation or filtration, then washed and dried. Optionally, the step of inducing crystallization can be done while stirring the reaction mixture prior to post crystallization aging.

Aging during stirring in an aging medium improves both the catalyst life and the POS when the sieve/catalyst is subsequently used in an oxygenates to olefins conversion process. The sieve is aged after it has been crystallized at a crystallization temperature. During aging the sieve mixture is stirred, preferably continuously stirred.

In an alternative embodiment, the sieve may be spray dried in the presence of a binder to make formulated catalyst particles and the sieve is aged in a slurrying fluid. In this embodiment, the synthesized sieve is re-dispersed in an aging medium and, following aging, the sieve is dried. The binder may be present in the aging medium or dispersed therein following aging. The resulting combination can then be formed into particles of the desired size and shape by spray drying.

There are many different binders that are useful in forming catalyst compositions. Non-limiting examples of binders that are useful alone or in combination include various types of hydrated alumina, silicas, and/or other inorganic oxide sols. The weight ratio of the binder to the molecular sieve may be from 0.1 to 0.5, such as from 0.1 to less than 0.5, for example from 0.11 to 0.48, conveniently from 0.12 to 0.45, typically from 0.13 to less than 0.45, and particularly from 0.15 to 0.4.

The binder may also comprise a matrix material comprising a clay or a clay-type composition, particularly having a low iron or titania content, and preferably comprises or is kaolin. Kaolin has been found to form a pumpable, high solids content slurry, to have a low fresh surface area, and to pack together easily due to its platelet structure. A preferred average particle size of the matrix material, preferably kaolin, is from 0.1 μm to 0.6 μm, with a D₉₀ particle size distribution of less than 1 μm.

In one embodiment of the invention, the crystallized silicoaluminophosphate molecular sieve has a crystal size distribution such that its average crystal size is at least 0.1 μm, preferably at least 1 μm, for example at least 3 μm.

As used herein, the term “average crystal size,” in reference to a crystal size distribution, should be understood to refer to a measurement on a representative sample or an average of multiple samples that together form a representative sample. Average crystal size can be measured by SEM, in which case the crystal size of at least 30 crystals must be measured in order to obtain an average crystal size, and/or average crystal size can be measured by a volumetric laser sizing instrument, in which case the measured d₅₀ of the sample(s) can represent the average crystal size, provided that the crystals are not agglomerated. Care should be taken that the values obtained by laser sizing match the crystal size as determined via SEM. It should also be understood that, while many of the crystals dealt with herein are relatively uniform (for instance, very close to cubic, thus having little difference between diameter measured along length, height, or width, e.g., when viewed in an SEM), the “average crystal size” represents the longest distance along one of the three-dimensional orthogonal axes (e.g., longest of length, width/diameter, and height, but not diagonal, in a cube, rectangle, parallelogram, ellipse, cylinder, frusto-cone, platelet, spheroid, or rhombus, or the like).

During aging of the crystalline molecular sieve, the aging temperature can be lower than the crystallization temperature. Preferably, the aging temperature is at least 20° C. lower than the crystallization temperature, more preferably at least 30° C. lower or at least 50° C. lower than the crystallization temperature. The sieve can be aged from 15 minutes to 120 hours, for example up to 60 hours, up to 48 hours, up to 8 hours, or up to 4 hours, depending on practical concerns relating to synthesis timing, cost efficiency, manufacture schedules, or the like. When the sieve is aged too long, a viscous slurry can sometimes result. When this happens, the sieve crystals cannot be recovered and are typically ineffective as a catalyst. The maximum duration for aging a particular sieve depends on the specific conditions, which can include aging medium, aging temperature, and type of sieve.

In another embodiment, one or more of the following are satisfied: the source of aluminum comprises alumina; the source of phosphorus comprises phosphoric acid; the source of silicon can include an organosilicate comprising a tetraalkylorthosilicate; and the at least one organic template comprises N,N dimethylcyclohexylamine.

The product of the crystallization is an aluminophosphate or silicoaluminophosphate containing a CHA framework-type molecular sieve having an X-ray diffraction pattern including at least the d-spacings shown in Table 1 below:

TABLE 1

| | Relative Intensities |
|---|---|
| d (A) | I/Io (%) |
| 9.26 | 100 |
| 6.30 | 20 |
| 5.64 | 15 |
| 5.51 | 57 |
| 4.96 | 25 |
| 4.92 | 27 |
| 4.29 | 76 |
| 4.18 | 21 |
| 3.55 | 32 |
| 3.50 | 20 |
| 3.42 | 10 |
| 2.91 | 22 |
| 2.88 | 26 |
| 2.87 | 19 |

Although the crystallization product is normally a single phase CHA framework-type molecular sieve, in some cases the product may contain an intergrowth of a CHA framework-type molecular sieve with, for example an AEI framework-type molecular sieve or small amounts of other crystalline phases, such as APC and/or AFI framework-type molecular sieves.

As a result of the crystallization process, the recovered crystalline product contains within its pores at least a portion of the organic directing agent used in the synthesis. In a preferred embodiment, activation is performed in such a manner that the organic directing agent is removed from the molecular sieve, leaving active catalytic sites within the microporous channels of the molecular sieve open for contact with a feedstock. The activation process is typically accomplished by calcining, or essentially heating the molecular sieve comprising the template at a temperature of from 200° C. to 800° C. in the presence of an oxygen-containing gas. In some cases, it may be desirable to heat the molecular sieve in an environment having a low or zero oxygen concentration. This type of process can be used for partial or complete removal of the organic directing agent from the intracrystalline pore system.

Once the crystalline product has been activated, it can be formulated into a catalyst composition by combination with other materials, such as binders and/or matrix materials, which provide additional hardness or catalytic activity to the finished catalyst.

Materials which can be blended with the present molecular sieve material include a large variety of inert and catalytically active materials. These materials include compositions such as kaolin and other clays, various forms of rare earth metals, other non-zeolite catalyst components, zeolite catalyst components, alumina or alumina sol, titania, zirconia, quartz, silica or silica sol, and mixtures thereof. These components are also effective in reducing overall catalyst cost, acting as a thermal sink to assist in heat shielding the catalyst during regeneration, densifying the catalyst and increasing catalyst strength. When blended with such components, the amount of present CHA-containing crystalline material contained in the final catalyst product ranges from 10 to 90 weight percent of the total catalyst, preferably 20 to 80 weight percent of the total catalyst.

The CHA framework type crystalline material produced by the present process can be used to dry gases and liquids; for selective molecular separation based on size and polar properties; as an ion-exchanger; as a chemical carrier; in gas chromatography; and as a catalyst in organic conversion reactions. Examples of suitable catalytic uses of the CHA framework type crystalline material described herein include (a) hydrocracking of heavy petroleum residual feedstocks, cyclic stocks and other hydrocrackate charge stocks, normally in the presence of a hydrogenation component selected from Groups 6 and 8-10 of the Periodic Table of Elements; (b) dewaxing, including isomerization dewaxing, to selectively remove straight chain paraffins from hydrocarbon feedstocks typically boiling above 177° C., including raffinates and lubricating oil basestocks; (c) catalytic cracking of hydrocarbon feedstocks, such as naphthas, gas oils, and residual oils, normally in the presence of a large pore cracking catalyst, such as zeolite Y; (d) oligomerization of straight and branched chain olefins having from 2-21, preferably 2-5, carbon atoms, to produce medium to heavy olefins which are useful for both fuels, e.g., gasoline or a gasoline blending stock, and chemicals; (e) isomerization of olefins, particularly olefins having 4-6 carbon atoms, and especially normal butene to produce iso-olefins; (f) upgrading of lower alkanes, such as methane, to higher hydrocarbons, such as ethylene and benzene; (g) disproportionation of alkylaromatic hydrocarbons, such as toluene, to produce dialkylaromatic hydrocarbons, such as xylenes; (h) alkylation of aromatic hydrocarbons, such as benzene, with olefins, such as ethylene and propylene, to produce alkylated aromatics, such as ethylbenzene and cumene; (i) isomerization of dialkylaromatic hydrocarbons, such as xylenes; (j) catalytic reduction of nitrogen oxides; and (k) synthesis of monoalkylamines and dialkylamines.

In particular, the CHA framework type crystalline material produced by the present process is useful as a catalyst in the conversion of oxygenates to one or more olefins, particularly ethylene and propylene. As used herein, the term “oxygenates” is defined to include, but is not necessarily limited to, aliphatic alcohols, ethers, carbonyl compounds (aldehydes, ketones, carboxylic acids, carbonates, and the like), and also compounds containing hetero-atoms, such as, halides, mercaptans, sulfides, amines, and mixtures thereof. The aliphatic moiety will normally contain from 1-10 carbon atoms, such as from 1-4 carbon atoms.

Representative oxygenates include lower straight chain or branched aliphatic alcohols, their unsaturated counterparts, and their nitrogen, halogen, and sulfur analogues. Examples of suitable oxygenate compounds can include, but are not necessarily limited to: methanol; ethanol; n-propanol; isopropanol; C₄ to C₁₀ alcohols; methyl ethyl ether; dimethyl ether; diethyl ether; di-isopropyl ether; methyl mercaptan; methyl sulfide; methyl amine; ethyl mercaptan; di-ethyl sulfide; di-ethyl amine; ethyl chloride; formaldehyde; di-methyl carbonate; di-methyl ketone; acetic acid; n-alkyl amines; n-alkyl halides; n-alkyl sulfides having n-alkyl groups comprising from 3-10 carbon atoms; and the like; and mixtures thereof Particularly suitable oxygenate compounds are methanol, dimethyl ether, and mixtures thereof, and most preferably comprise methanol. As used herein, the term “oxygenate” designates only the organic material used as the feed. The total charge of feed to the reaction zone may contain additional compounds, such as diluents.

In one embodiment of the oxygenate conversion process, a feedstock comprising an organic oxygenate, optionally with one or more diluents, is contacted in the vapor phase in a reaction zone with a catalyst comprising the present molecular sieve at effective process conditions so as to produce the desired olefins. Alternatively, the process may be carried out in a liquid or a mixed vapor/liquid phase. When the process is carried out in the liquid phase or a mixed vapor/liquid phase, different conversion rates and selectivities of feedstock-to-product may result depending upon the catalyst and the reaction conditions.

When present, the diluent(s) is(are) generally non-reactive to the feedstock or molecular sieve catalyst composition and is typically used to reduce the concentration of the oxygenate in the feedstock. Non-limiting examples of suitable diluents include helium, argon, nitrogen, carbon monoxide, carbon dioxide, water, essentially non-reactive paraffins (especially alkanes such as methane, ethane, and propane), essentially non-reactive aromatic compounds, and mixtures thereof. The most preferred diluents include water and nitrogen, with water being particularly preferred. Diluent(s) may comprise from 1 mol % to 99 mol % of the total feed mixture.

The temperature employed in the oxygenate conversion process may vary over a wide range, such as from 200° C. to 1000° C., for example from 250° C. to 800° C., including from 250° C. to 750° C., conveniently from 300° C. to 650° C., typically from 350° C. to 600° C. and particularly from 400° C. to 600° C.

Light olefin products will form, although not necessarily in optimum amounts, at a wide range of pressures, including but not limited to autogenous pressures and pressures in the range from 0.1 kPa to 10 MPa. Conveniently, the pressure can be in the range from 7 kPa to 5 MPa, such as from 50 kPa to 1 MPa. The foregoing pressures are exclusive of diluents, if any are present, and refer to the partial pressure of the feedstock as it relates to oxygenate compounds and/or mixtures thereof. Lower and upper extremes of pressure may adversely affect selectivity, conversion, coking rate, and/or reaction rate; however, light olefins such as ethylene and/or propylene still may form.

In a preferred embodiment, the method of converting hydrocarbons into olefins according to the invention comprises: (a) preparing a silicoaluminophosphate molecular sieve according to the methods disclosed hereinabove; (b) formulating said silicoaluminophosphate molecular sieve, along with a binder and optionally a matrix material, into a silicoaluminophosphate molecular sieve catalyst composition, typically comprising from at least 10% to 50% molecular sieve; and (c) contacting said catalyst composition with a hydrocarbon feed under conditions sufficient to convert said hydrocarbon feed into a product comprising predominantly one or more olefins. Preferably, the hydrocarbon feed is an oxygenate-containing feed comprising methanol, dimethylether, or a combination thereof, and the one or more olefins typically comprises ethylene, propylene, or a combination thereof.

A wide range of weight hourly space velocities (WHSV) for the feedstock will function in the oxygenate conversion process. WHSV is defined as weight of feed (excluding diluents) per hour per weight of a total reaction volume of molecular sieve catalyst (excluding inert and/or fillers). The WHSV generally should be in the range from 0.01 hr⁻¹ to 500 hr⁻¹, such from 0.5 hr⁻¹ to 300 hr⁻¹, for example from 0.1 hr⁻¹ to 200 hr⁻¹.

A practical embodiment of a reactor system for the oxygenate conversion process is a circulating fluid bed reactor with continuous regeneration. Fixed beds are generally not preferred for the process, because oxygenate-to-olefin conversion is a highly exothermic process that requires several stages with intercoolers or other cooling devices. The reaction also results in a high pressure drop, due to the production of low pressure, low density gas.

Because the catalyst typically needs to be regenerated frequently, the reactor should preferably allow easy removal of at least a portion of the catalyst to a regenerator, where the catalyst can be subjected to a regeneration medium, such as a gas comprising oxygen, for example air, to burn off coke from the catalyst, which should restore at least some of the catalyst activity. The conditions of temperature, oxygen partial pressure, and residence time in the regenerator can typically be selected to achieve a coke content on regenerated catalyst of less than 1 wt %, for example less than 0.5 wt %. At least a portion of the regenerated catalyst should be returned to the reactor.

In a preferred embodiment, the method of forming an olefin-containing polymer product comprises: (a) preparing a silicoaluminophosphate molecular sieve according to the methods described hereinabove; (b) formulating said silicoaluminophosphate molecular sieve, along with a binder and optionally a matrix material, into a silicoaluminophosphate molecular sieve catalyst composition comprising from at least 10% to 50% molecular sieve; (c) contacting said catalyst composition with a hydrocarbon feed under conditions sufficient to convert said hydrocarbon feed into a product comprising predominantly one or more; and (d) polymerizing at least one of the one or more olefins, optionally with one or more other comonomers and optionally (but preferably) in the presence of a polymerization catalyst, under conditions sufficient to form an olefin-containing (co)polymer.

Preferably, the hydrocarbon feed is an oxygenate-containing feed comprising methanol, dimethylether, or a combination thereof, the one or more olefins typically comprises ethylene, propylene, or a combination thereof, and the olefin-containing (co)polymer is an ethylene-containing (co)polymer, a propylene-containing (co)polymer, or a copolymer, mixture, or blend thereof

Additionally or alternately, the invention can include the following embodiments.

Embodiment 1. A method of preparing a crystalline microporous silicoalumino-phosphate molecular sieve comprising:
(a) providing a reaction mixture having a molar composition within the following ranges:
   P:Al from 0.75 to 1.25,
   Si Al₂ from 0.01 to 0.32,
   H₂O:Al₂ from 25 to 50, and
   R:Al₂ from 1 to 3,
      where R designates a structure directing agent, and wherein the molar ratios for the aluminum, phosphorus, and silicon sources are calculated based on the oxide forms;
(b) heating the mixture to a crystallization temperature of between 150° C. to 250° C. to form the crystalline molecular sieve within the reaction mixture; and
(c) following crystallization in step b), aging the crystalline molecular sieve in an alkaline liquid at a temperature below the crystallization temperature from 0.5 hour for up to 120 hours.

Embodiment 2. The method of embodiment 1, wherein the alkaline liquid comprises the reaction mixture.

Embodiment 3. The method of embodiment 1 or embodiment 2, wherein, following aging, the sieve is dried.

Embodiment 4. The method of embodiment 3, wherein, prior to drying, the sieve is washed.

Embodiment 5. The method of any of the previous embodiments, wherein the alkaline liquid is a washing fluid and/or slurrying fluid.

Embodiment 6. The method of any of the previous embodiments, wherein the mixture is stirred during aging.

Embodiment 7. The method of any of the previous embodiments, wherein the crystallized molecular sieve has an average crystal size greater than 1 μm, preferably greater than 2.5 μm or greater than 3.0 μm.

Embodiment 8. The method of any of the previous embodiments, wherein the mixture is aged at a temperature which is at least 30° C. below, preferably at least 50° C. below, the crystallization temperature.

Embodiment 9. The method of any of the previous embodiments, wherein the source of silica comprises an inorganic silicon source such as a colloidal silica.

Embodiment 10. The method of any of embodiments 1-8, wherein the source of silica comprises an organic silicon source such as a tetraalkyl orthosilicate.

Embodiment 11. The method of any of the previous embodiments, wherein the molecular sieve has a CHA framework.

Embodiment 12. The method of any of the previous embodiments, wherein the reaction mixture comprises a seed having a framework type of CHA, AEI, AFX, LEV, an intergrowth thereof, or a combination thereof.

Embodiment 13. A method of converting oxygenates into one or more olefins comprising:

(a) preparing a silicoaluminophosphate molecular sieve according to the method of any of the previous embodiments;

(b) formulating said silicoaluminophosphate molecular sieve, along with a binder and optionally a matrix material, into a silicoaluminophosphate molecular sieve catalyst composition comprising from at least 10% to 50% molecular sieve; and

(c) contacting said catalyst composition with an oxygenate feed under conditions sufficient to convert said oxygenate feed into a product comprising predominantly one or more olefins.

Embodiment 14. A method of forming an olefin-containing polymer product comprising:

(a) preparing a silicoaluminophosphate molecular sieve according to the method of any of embodiments 1-12;

(b) formulating said silicoaluminophosphate molecular sieve, along with a binder and optionally a matrix material, into a silicoaluminophosphate molecular sieve catalyst composition comprising from at least 10% to 50% molecular sieve;

(c) contacting said catalyst composition with an oxygenate feed under conditions sufficient to convert said oxygenate feed into a product comprising predominantly one or more olefins; and

(d) polymerizing at least one of the one or more olefins, optionally with one or more other comonomers and optionally in the presence of a polymerization catalyst, under conditions sufficient to form an olefin-containing (co)polymer.

Embodiment 15. The method of embodiment 13 or embodiment 14, wherein the oxygenate feed comprises methanol, dimethylether, or a combination thereof, wherein the one or more olefins comprises ethylene, propylene, or a combination thereof, and wherein, when applicable, the olefin-containing (co)polymer is an ethylene-containing (co)polymer, a propylene-containing (co)polymer, or a copolymer, mixture, or blend thereof.

### EXAMPLES

Aging affects both catalyst life, and catalyst activity as we will now show with reference to the below Examples.

### Example 1

A synthesis mixture having a molar composition of about 0.15 SiO₂:P₂O₅:Al₂O₃:2 DMCHA:40 H₂O, as well as 100 wt ppm (wppm) seeds, was prepared according to the following procedure. A solution of phosphoric acid was prepared by combining phosphoric acid [Acros, 85%] and water. To this solution was added the appropriate amount of Condea Pural SB [Sasol, 75.6 wt % Al₂O₃], and the slurry was stirred for about 1 hour at about 10° C. To this mixture was added the appropriate amount of Ludox AS40 [ammonium stabilized silica sol containing 40 wt % SiO₂, from Grace Nev.]. Then the appropriate amount of dimethylcyclohexylamine [DMCHA, 99%, from Purum Fluka] was added. This mixture was stirred for about 10 minutes before the seeds (SAPO-34 seeds) were added. The final mixture was transferred to an autoclave which was heated, while stirring, to about 170° C. with a heat-up rate of about 20° C./hr. After about 24 hours this temperature, the autoclave was cooled to approximately room temperature, and the mixture was aged for varying periods of time without mixing. The yield was determined by weighing the dried solids and dividing this weight by the weight of the initial synthesis mixture. The so-calculated yield, prior to aging, was determined to be about 16.5 wt %.

After aging, the solids were washed with demineralized water and dried at about 120° C. The phase purity of the sample was determined by X-ray diffraction and was characterized substantially by the d-spacings shown in Table 1 above.

### Example 2

Similar to the mixture in Example 1 above, a synthesis mixture having a molar composition of about 0.15 SiO₂:P₂O₅:Al₂O₃:2 DMCHA:40 H₂O, as well as 100 wppm seeds, was prepared according to the following procedure. A solution of phosphoric acid was prepared by combining phosphoric acid [Acros, 85%] and water. To this solution was added the appropriate amount of Condea Pural SB [Sasol, 75.6 wt % Al₂O₃], and the slurry was stirred for about 1 hour at about 10° C. To this mixture was added the appropriate amount of tetraethylorthosilicate [TEOS, 98%, from Aldrich]. Then the appropriate amount of dimethylcyclohexylamine [DMCHA, 99%, from Purum Fluka] was added. This mixture was stirred for about 10 minutes before the seeds (SAPO-34 seeds) were added. The final mixture was transferred to an autoclave which was heated, while stirring, to about 170° C. with a heat-up rate of about 20° C./hr. After about 24 hours this temperature, the autoclave was cooled to approximately room temperature, and the mixture was aged for varying periods of time without mixing. The yield was determined by weighing the dried solids and dividing this weight by the weight of the initial synthesis mixture. The so-calculated yield, prior to aging, was determined to be about 10.5 wt %.

After aging, the solids were washed with demineralized water and dried at about 120° C. The phase purity of the sample was determined by X-ray diffraction and was characterized substantially by the d-spacings shown in Table 1 above.

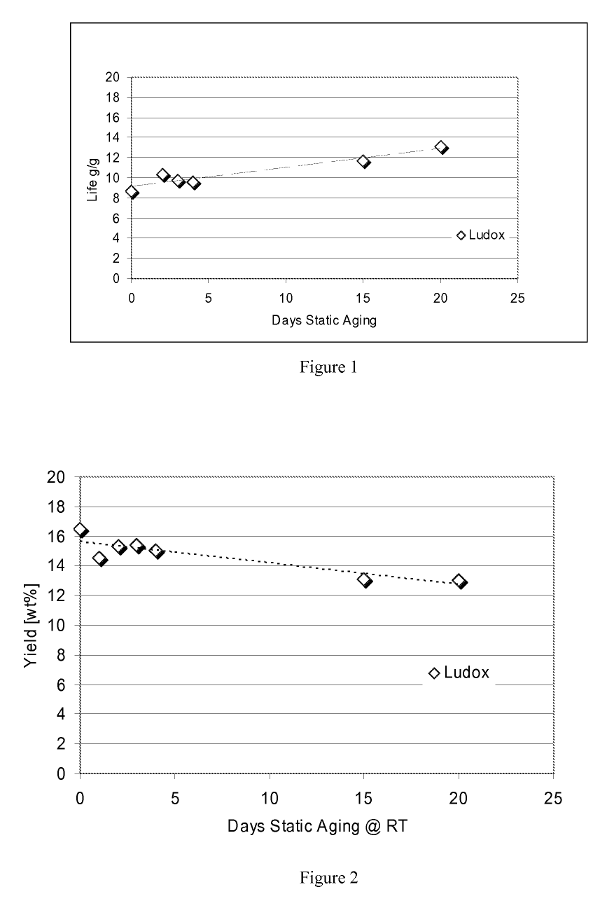

FIG. 1 and FIG. 2 present the relationship between static aging and catalyst life and between static aging and yield, respectively, for the catalyst made in accordance with Example 1. FIG. 1 shows that, for a catalyst prepared from a colloidal silica source, catalyst life appears to increase as the synthesis mixture is aged without stirring. FIG. 2 further shows that the yield appears to decrease with increasing time of static aging.

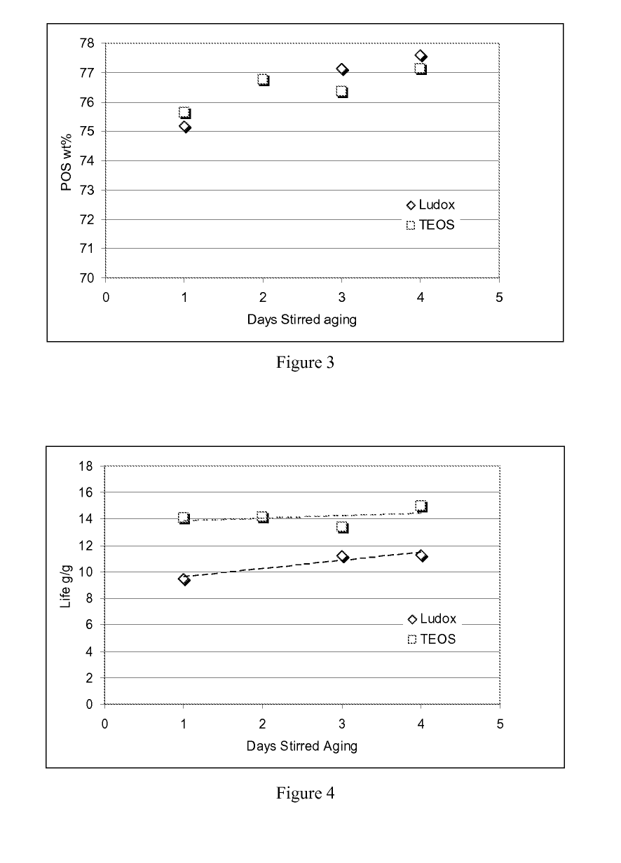

Example 1 was prepared from Ludox as a silica source, whereas Example 2 was prepared from TEOS as a silica source. The product sieve of each of Examples 1 and 2 was pelletized and calcined at about 600° C. for about 4 hours in air, and was then tested for its methanol-to-olefins (MTO) conversion performance under the following conditions: reaction temperature of about 500° C.; WHSV of about 100 g MeOH/g sieve/hr; and total pressure of about 25 psig (about 173 kPag). FIG. 3 showed an increase in POS for both catalysts with stirred aging. FIG. 4 further showed an increase in catalyst life with increased stirred aging time, although the improvement for the TEOS-based sample was relatively modest. The catalyst life, or CMCPS, in these Figures is defined as mass (g) of oxygenate (in this case, methanol) converted per mass (g) of sieve, before the oxygenate conversion falls below 10%. Longer lifetimes are generally preferred, as they also tend to correspond to higher average kinetic rates and/or better catalyst utilization.

The Examples show that aging can have a beneficial effect on both POS and on catalyst life, although catalyst yield can be somewhat compromised.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

What is claimed is:

1. A method of preparing a crystalline microporous silicoaluminophosphate molecular sieve comprising:
(a) providing a reaction mixture having a molar composition within the following ranges:
P:Al from 0.75 to 1.25,
Si:Al₂ from 0.01 to 0.32,
H₂O:Al₂ from 25 to 50, and
R:Al₂ from 1 to 3,
where R designates a structure directing agent, and wherein the molar ratios for the aluminum, phosphorus, and silicon sources are calculated based on the oxide forms;
(b) heating the mixture to a crystallization temperature of between 150° C. to 250° C. to form the crystalline molecular sieve within the reaction mixture; and
(c) following crystallization in step b), during stirring, aging the crystalline molecular sieve in an alkaline liquid at a temperature below the crystallization temperature from 0.5 hour for up to 120 hours.

2. The method of claim 1, wherein the alkaline liquid comprises the reaction mixture.

3. The method of claim 1, wherein, following aging, the sieve is dried.

4. The method of claim 3, wherein, prior to drying, the sieve is washed.

5. The method of claim 3, wherein the alkaline liquid is a washing fluid and/or slurrying fluid.

6. The method of claim 1, wherein the mixture is stirred continuously during aging.

7. The method of claim 1, wherein the crystallized molecular sieve has an average crystal size greater than 2.0 μm.

8. The method of claim 1, wherein the mixture is aged at a temperature which is at least 30° C. below the crystallization temperature.

9. The method of claim 1, wherein the source of silica comprises a colloidal silica.

10. The method of claim 1, wherein the source of silica comprises an organic silicon source.

11. The method of claim 1, wherein the molecular sieve has a CHA framework.

12. The method of claim 1, wherein the reaction mixture comprises a seed having a framework type of CHA, AEI, AFX, LEV, an intergrowth thereof, or a combination thereof.

13. A method of converting oxygenates into one or more olefins comprising:
(a) preparing a silicoaluminophosphate molecular sieve according to the method of claim 1;
(b) formulating said silicoaluminophosphate molecular sieve, along with a binder and optionally a matrix material, into a silicoaluminophosphate molecular sieve catalyst composition comprising from at least 10% to 50% molecular sieve; and
(c) contacting said catalyst composition with an oxygenate feed under conditions sufficient to convert said oxygenate feed into a product comprising predominantly one or more olefins.

14. The method of claim 13, wherein the oxygenate feed comprises methanol, dimethylether, or a combination thereof, and wherein the one or more olefins comprises ethylene, propylene, or a combination thereof.

15. A method of forming an olefin-containing polymer product comprising:
(a) preparing a silicoaluminophosphate molecular sieve according to the method of claim 1;
(b) formulating said silicoaluminophosphate molecular sieve, along with a binder and optionally a matrix material, into a silicoaluminophosphate molecular sieve catalyst composition comprising from at least 10% to 50% molecular sieve;
(c) contacting said catalyst composition with an oxygenate feed under conditions sufficient to convert said oxygenate feed into a product comprising predominantly one or more olefins; and
(d) polymerizing at least one of the one or more olefins, optionally with one or more other comonomers and optionally in the presence of a polymerization catalyst, under conditions sufficient to form an olefin-containing (co)polymer.

16. The method of claim 15, wherein the oxygenate feed comprises methanol, dimethylether, or a combination thereof, wherein the one or more olefins comprises ethylene, propylene, or a combination thereof, and wherein the olefin-containing (co)polymer is an ethylene-containing (co)polymer, a propylene-containing (co)polymer, or a copolymer, mixture, or blend thereof.
